(19) United States
(12) Patent Application Publication
(10) Pub. No.: US 2022/0053838 A1
(43) Pub. Date: Feb. 24, 2022

(54) METHOD AND APPARATUS FOR INFANT POSITIONING FOR REDUCTION OF SUDDEN INFANT DEATH AND PLAGIOCEPHALY

(71) Applicant: Wayne Kennard Scott, JR., Sachse, TX (US)

(72) Inventor: Wayne Kennard Scott, JR., Sachse, TX (US)

(21) Appl. No.: 17/274,874

(22) PCT Filed: Sep. 17, 2019
(86) PCT No.: PCT/US2019/051570

§ 371 Date: Mar. 10, 2021

Related U.S. Application Data
(60) Provisional application No. 62/846,336, filed on May 10, 2019.
(60) Provisional application No. 62/732,901, filed on Sep. 18, 2018.

(51) Int. Cl. A41B 13/06; A61F 5/37

(52) U.S. Cl. CPC A41B 13/065; A61F 5/37

(57) ABSTRACT

A garment includes a body. Right and left sleeves are coupled to the body. The right sleeve and the left sleeve each include a first fastener that is operable to secure the right sleeve and the left sleeve to the body, respectively. Right and left legs are coupled to the body. The right leg and the left leg each include a second fastener that is operable to secure the right leg to the left leg. Third fasteners are arranged on a back portion of the body.

### CROSS-REFERENCE TO RELATED APPLICATIONS

This application claims priority to and incorporates by reference the disclosures of U.S. Provisional Patent Application No. 62/732,901, filed on Sep. 18, 2018 and U.S. Provisional Patent Application No. 62/846,336, filed on May 10, 2019.

### TECHNICAL FIELD

The present disclosure relates generally to child-care devices and more particularly, but not by way of limitation, to a method and systems for facilitating the securement of an infant in an appropriate sleeping position.

### BACKGROUND

This section provides background information to facilitate a better understanding of the various aspects of the disclosure. It should be understood that the statements in this section of this document are to be read in this light, and not as admissions of prior art.

Sudden Infant Death Syndrome (“SIDS”) is defined as the sudden death of an infant under one year of age, which remains unexplained after a thorough case investigation. SIDS is a leading cause of death in infants between one month and one year of age. Recent public-awareness campaigns aimed at promoting safe infant sleeping positions have proven successful in reducing the incidence of SIDS. Two such safe sleeping positions commonly advocated are a back-sleeping position and a side-sleeping position. The back-sleeping position, in particular, has led to an increase in “flat head” also referred to as “brachycephaly or plagiocephaly” caused by extended times the infant spends on its back.

### SUMMARY

This summary is provided to introduce a selection of concepts that are further described below in the detailed description. This summary is not intended to identify key or essential features of the claimed subject matter, nor is it to be used as an aid in limiting the scope of the claimed subject matter.

In one aspect, the present disclosure relates to an infant garment. The garment includes a body. Right and left sleeves are coupled to the body. The right sleeve and the left sleeve each include a first fastener that is operable to secure the right sleeve and the left sleeve to the body, respectively. Right and left legs are coupled to the body. The right leg and the left leg each include a second fastener that is operable to secure the right leg to the left leg. Third fasteners are arranged on a back portion of the body.

In one aspect, the present disclosure relates to an infant-positioning system. The infant positioning system includes a base. A sleeping surface is coupled to the base. A stabilizer wing is slidably or hingedly coupled to the base. The stabilizer wing is extendable to increase a support area of the base. A fastener facilitates attachment of the base to a garment.

In one aspect, the present disclosure relates to an infant swaddling system. The infant swaddling system includes a garment. The garment includes first fasteners to secure the garment about a torso of the infant and second fasteners disposed on a back region of the garment. The system includes a blanket. The blanket includes third fasteners that engage the second fasteners disposed on the garment. Oppositely-disposed side edges are disposed outwardly of the third fasteners. A lower region is disposed below the third fasteners. The lower region and the oppositely-disposed side edges are selectively foldable about the infant so as to swaddle the infant.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion.

FIG. 1 is a top plan view of a modular base assembly with stabilizing wings extended according to aspects of the disclosure;

FIG. 2 is a top plan view of a modular base assembly with stabilizing wings retracted according to aspects of the disclosure;

FIG. 3 is a cross-sectional view of a modular base assembly with a positioning insert that creates an angular sleeping surface coupled thereto according to aspects of the disclosure;

FIG. 4 is a perspective view of a modular base assembly with a positioning insert that creates an angular sleeping surface coupled thereto and stabilizing wings extended according to aspects of the disclosure;

FIG. 5A is a perspective view of a modular base assembly with a flat sleeping surface coupled thereto and stabilizing wings retracted according to aspects of the disclosure;

FIG. 5B is a cross-sectional view of the flat sleeping surface of FIG. 5A;

FIG. 6A is a front view of an infant bodice restraint article according to aspects of the disclosure in an open position around an infant;

FIG. 6B is a front view of an infant bodice restraint article according to aspects of the disclosure in a closed position around an infant;

FIG. 6C is a rear view of an infant bodice restraint article according to aspects of the disclosure in a closed position around an infant;

FIG. 6D is a front view of an infant garment according to aspects of the disclosure;

FIG. 7A is a front view of a swaddle wrap in an open position and placed around an infant;

FIG. 7B is a front, partially-folded view of the swaddle wrap of FIG. 7A;

FIG. 7C is a front, partially-folded view of the swaddle wrap of FIG. 7A;

FIG. 7D is a front, folded, view of the swaddle wrap of FIG. 7A;

FIG. 7E is a rear, folded, view of the swaddle wrap of FIG. 7A showing fasteners;

FIG. 7F is a perspective view of the swaddle wrap of FIG. 7A coupled to a back support;

FIG. 7G is a perspective view of the swaddle wrap of FIG. 7A coupled to a back support and stabilizer;

FIG. 7H is a perspective view of the swaddle wrap of FIG. 7A coupled to an angular sleeping surface;

FIG. 8A is a rear view of a swaddling garment with fasteners open according to aspects of the disclosure;

FIG. 8B is a rear view of a swaddling garment with fasteners closed according to aspects of the disclosure;

FIG. 8C is a front view of a swaddling garment with fasteners closed according to aspects of the disclosure;

FIG. 8D is a front view of a swaddling garment with fasteners closed according to aspects of the disclosure;

FIGS. 9A-9D illustrate use of the swaddling garment an outer swaddling wrap;

FIG. 10A is a top view of a stabilizing frame in an extended position according to aspects of the disclosure;

FIG. 10B is a top view of the stabilizing frame in a retracted position according to aspects of the disclosure;

FIG. 10C is a top view of a swaddling garment coupled to a back support and a stabilizing frame according to aspects of the disclosure;

FIG. 10D is a top view of a swaddling garment coupled to a stabilizing frame according to aspects of the disclosure;

FIG. 11A is a rear view of an infant garment showing swaddling flaps in a stored position according to aspects of the disclosure;

FIG. 11B is a front view of the infant garment of FIG. 11A in an open position according to aspects of the disclosure;

FIG. 11C is a front view of the infant garment of FIG. 11A showing fastening of sleeves to a torso region according to aspects of the disclosure;

FIG. 11D is a front view of the infant garment of FIG. 11A showing sleeves fastened to each other according to aspects of the disclosure;

FIG. 11E is a front view of the infant garment of FIG. 11A showing the swaddling flaps partially secured according to aspects of the disclosure;

FIG. 11F is a front view of the infant garment of FIG. 11A showing the swaddling flaps secured to each other in front of the infant garment according to aspects of the disclosure;

FIG. 11G is a front view of an infant garment illustrating a bottom fastener and opening in a closed position according to aspects of the disclosure;

FIG. 11H is a front view of the infant garment of FIG. 11G in an open position.

FIG. 11I is a front view of the infant garment of FIG. 11A showing open swaddling flaps according to aspects of the disclosure;

FIG. 12A is a front view of an infant garment according to aspects of the disclosure;

FIG. 12B is a rear view of the infant garment of FIG. 12A showing swaddling flaps in a stored position according to aspects of the disclosure;

FIG. 12C is a front view of the infant garment of FIG. 12A showing a vertically-oriented fastener in an open position according to aspects of the disclosure;

FIG. 12D is a front view of the infant garment of FIG. 12A showing the vertically-oriented fastener in an alternative open position according to aspects of the disclosure;

FIG. 12E is a front view of the infant garment of FIG. 12A showing sleeves joined to a torso region according to aspects of the disclosure;

FIG. 12F is a front view of the infant garment of FIG. 12A showing sleeves joined to each other according to aspects of the disclosure;

FIG. 12G is a front view of the infant garment of FIG. 12A showing the swaddling flaps partially secured according to aspects of the disclosure;

FIG. 12H is a front view of the infant garment of FIG. 12A showing the swaddling flaps secured to each other in front of the infant garment according to aspects of the disclosure;

FIG. 12I is a front view of the infant garment of FIG. 12A showing open swaddling flaps according to aspects of the disclosure;

FIG. 13A is a front view of an infant garment according to aspects of the disclosure;

FIG. 13B is a front view of the infant garment of FIG. 13A illustrating swaddling flaps and tether;

FIG. 13C is a front view of the infant garment of FIG. 13A illustrating the tether securing the swaddling flaps;

FIG. 13D is a front view of the infant garment of FIG. 13A illustrating the swaddling flaps being secured; and

FIG. 13E is a front view of the infant garment of FIG. 13A showing open swaddling flaps according to aspects of the disclosure.

### DETAILED DESCRIPTION

Various embodiments will now be described more fully with reference to the accompanying drawings. The disclosure may, however, be embodied in many different forms and should not be construed as limited to the embodiments set forth herein.

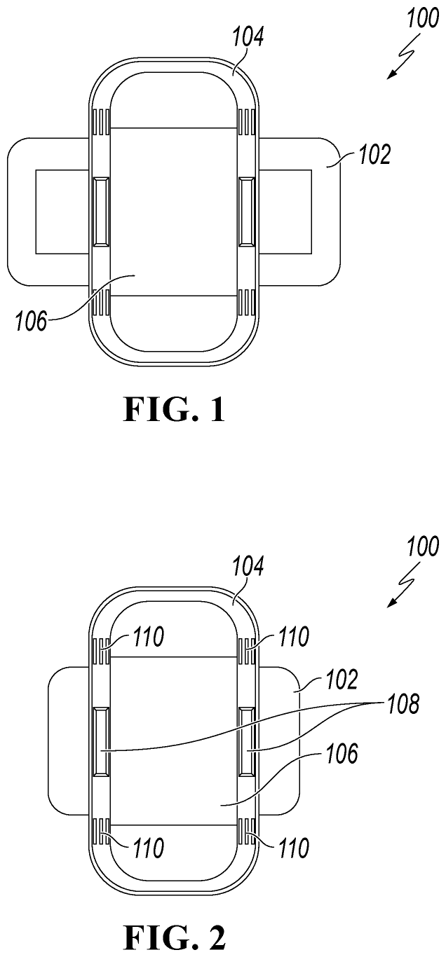

FIG. 1 is a top plan view of a modular base assembly 100 with stabilizer wings 102 extended. FIG. 2 is a top plan view of a modular base assembly 100 with stabilizer wings 102 retracted. Referring to FIGS. 1-2 collectively, the modular base assembly 100 includes a base 104 and a sleeping surface 106. In various embodiments, the base 104 is constructed of a rigid material such as, for example, plastic, wood, metal, and composite material and the sleeping surface 106 is constructed of a softer, pliable material such as, for example foam or a bladder filled with gas, liquid, or gel. In various embodiments, the material of the sleeping surface 106 promotes comfort when occupied by an infant.

Still referring to FIGS. 1-2, stabilizer wings 102 are coupled to the base 104. In various embodiments, the stabilizer wings 102 are slidably adjustable between an extended position and a retracted position. In other embodiments, the stabilizer wings 102 could be hingedly coupled to the base 104. FIGS. 1-2 illustrate by way of example, a modular base assembly 100 having two stabilizer wings 102; however, in other embodiments, the modular base assembly 100 could have any number of stabilizer wings 102. In various embodiments, however, the stabilizer wings 102 could be omitted. When in the extended position, the stabilizer wings 102 enlarge a support area of the modular base assembly 100 thereby causing the modular base assembly 100 to resist rolling when occupied by an infant.

Still referring to FIGS. 1-2, the sleeping surface 106 includes first attachment points 108. In various embodiments, the first attachment points 108 may include, for example, buttons, snaps, hook and pile fasteners, interlocking fasteners such as, for example, zippers, or slots through which a securement strap could be received. The first attachment points 108 facilitate attachment of the modular base assembly 100 to another device such as, for example, a swing, a cradle, a rocker, a bassinette, a play yard, or any other device in which an infant might be placed in a reclined or supine position. The sleeping surface 106 may also include second attachment points 110. In various embodiments, the second attachment points 110 may include, for example, snaps, hook and pile fasteners, interlocking fasteners such as, for example, buttons, zippers, or slots through which a securement strap could be received. The second attachment points 110 facilitate attachment of the sleeping surface 106 to an infant securement device as will be discussed in more detail hereinbelow.

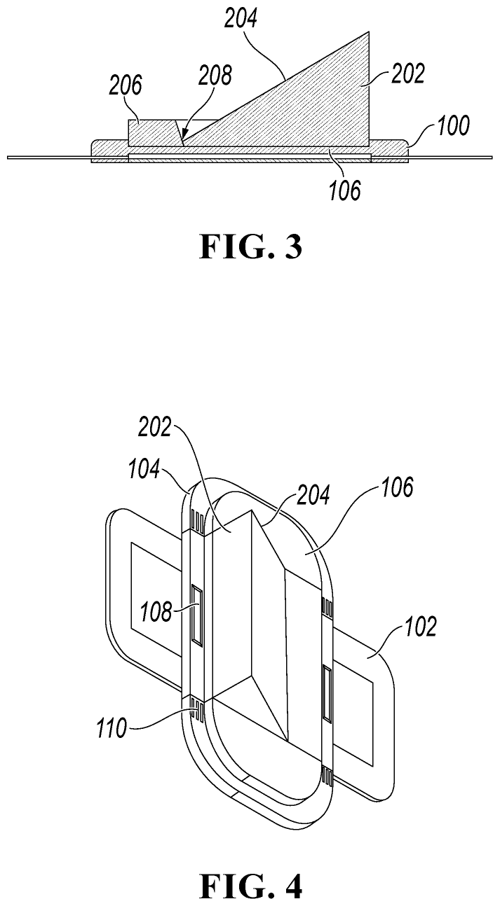

FIG. 3 is a cross-sectional view of the modular base assembly 100 with a positioning insert 202 that is illustrated by way of example as an angular sleeping block coupled thereto. FIG. 4 is a perspective view of the modular base assembly 100 with the positioning insert 202 coupled thereto and stabilizer wings 102 extended. Referring to FIGS. 3-4 collectively, the positioning insert 202 includes a sleeping surface 204 and a shoulder rest 206. In various embodiments, the positioning insert 202 could be a cylinder, a polygon, a wedge, a curved shape, or any combination thereof. In various embodiments, however, the shoulder rest 206 may be omitted. In various embodiments, the sleeping surface 204 is inclined relative to horizontal at an angle in a range of approximately 15 degrees to approximately 90 degrees. The sleeping surface 204 is of a width sufficient to accommodate an infant.

Still referring to FIGS. 3-4, the shoulder rest 206 includes a stop surface 208. In various embodiments, the stop surface 208 may be oriented at an angle opposite the inclination of the sleeping surface 204. The stop surface 208 may be inclined relative to horizontal at an angle in a range of approximately 90 degrees to approximately 15 degrees. By way of example, the shoulder rest is illustrated in FIGS. 3-4 as being a block; however, in other embodiments, the shoulder rest 206 could be a cylinder, a polygon, a wedge, or a curved shape. In use, the stop surface 208 abuts a shoulder of an infant and prevents the infant from sliding off the sleeping surface 204. In various embodiments, the positioning insert 202 is coupled to the sleeping surface 106 via connections such as, for example, buttons, straps, snaps, or hook and loop fasteners.

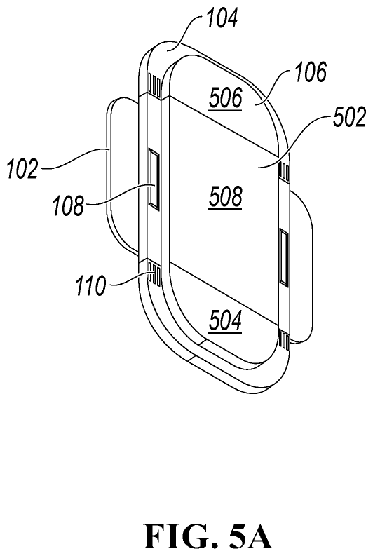

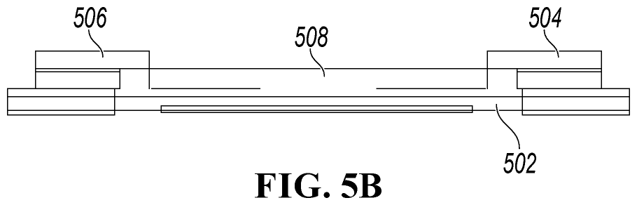

FIG. 5A is a perspective view of a modular base assembly 100 with a flat sleeping surface 502 coupled thereto and stabilizer wings 102 retracted. In various embodiments, the flat sleeping surface 502 may be coupled to the sleeping surface 106 via connections such as, for example, buttons, straps, snaps, or hook and loop fasteners. As shown in FIG. 5B, in various embodiments, the foot-rest area 504 and the head-rest area 506 may be slightly elevated relative to the torso area 508.

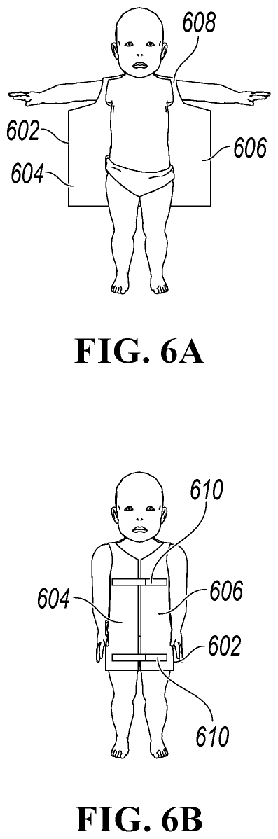

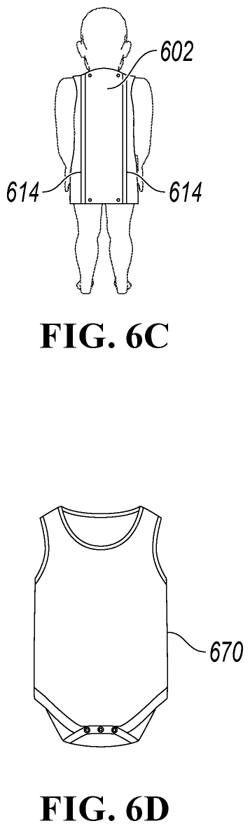

FIG. 6A is a front view of an infant bodice restraint article 602 in an open position around an infant. FIG. 6B is a front view of the infant bodice restraint article 602 in a closed position around the infant. FIG. 6C is a rear view of the infant bodice restraint article 602 in a closed position around an infant. Referring to FIGS. 6A-6C collectively, the infant bodice restraint article 602 includes a left side 604 and a right side 606. Arm holes 608 are formed in the left side 604 and the right side 606, respectively. In use, the left side 604 and the right side 606 close around a torso region of an infant. The left side 604 and the right side 606 are secured to each other by fasteners 610. In various embodiments, the fasteners 610 may include buttons, snaps, hook and pile fasteners, interlocking fasteners, or any combination thereof. In various embodiments, the infant bodice restraint article 602 may be size adjustable such that the left side 604 is secured to the right side 606 snugly about the torso region of the infant. By positioning the arms of the infant through the arm holes 608, the infant is prevented from turning in the infant bodice restraint article 602. As shown in FIG. 6C, a rear face 612 of the infant bodice restraint article 602 includes fasteners 614 to facilitate attachment of the infant bodice restraint article 602 to other accessories such as, for example, the modular base assembly 100, the positioning insert 202, the swaddle wrap 702, the back support 722, the stabilizer wings 724, the support frame 1002, or any combination thereof. FIG. 6C illustrates by way of example interlocking fasteners; however, in other embodiments, the fasteners 614 could be, for example, buttons, snaps, hook and pile fasteners, clips, interlocking fasteners, or any other type of fastener. In still other embodiments, the infant bodice restraint article 602 may be permanently attached to another accessory such as, for example, the modular base assembly 100, the positioning insert 202, the swaddle wrap 702, the back support 722, the attachable stabilizer wings 724 (discussed below), the support frame 1002, or any combination of the above. In various embodiments, the infant bodice restraint article 602 could be substituted with a infant garment 670 such as, for example, a onesie (shown in FIG. 6D). In various embodiments, the infant garment 670 may include arm and leg sleeves. The infant garment 670 would include the fasteners 614 to facilitate attachment to other accessories such as, for example, the modular base assembly 100, the positioning insert 202, the swaddle wrap 702, the back support 722, the attachable stabilizer wings 724, the support frame 1002, or any combination of the above. In other embodiments, the bodice restraint article 602 or the infant garment 670 may be permanently coupled to at least one of the modular base assembly 100, the positioning insert 202, the swaddle wrap 702, the back support 722, or the support frame 1002.

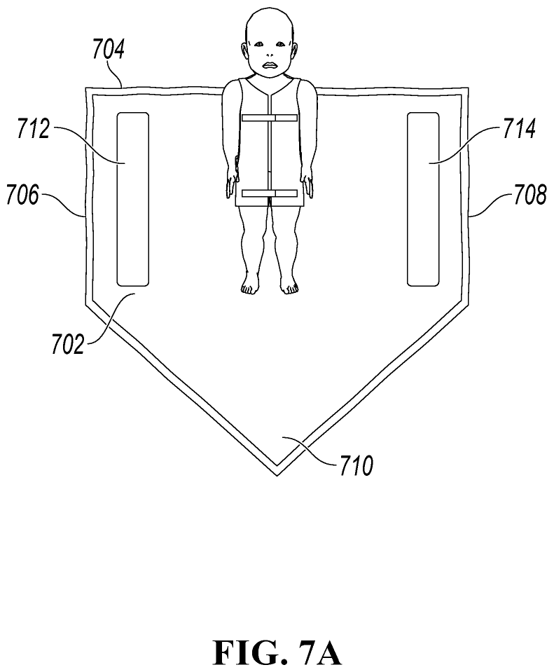

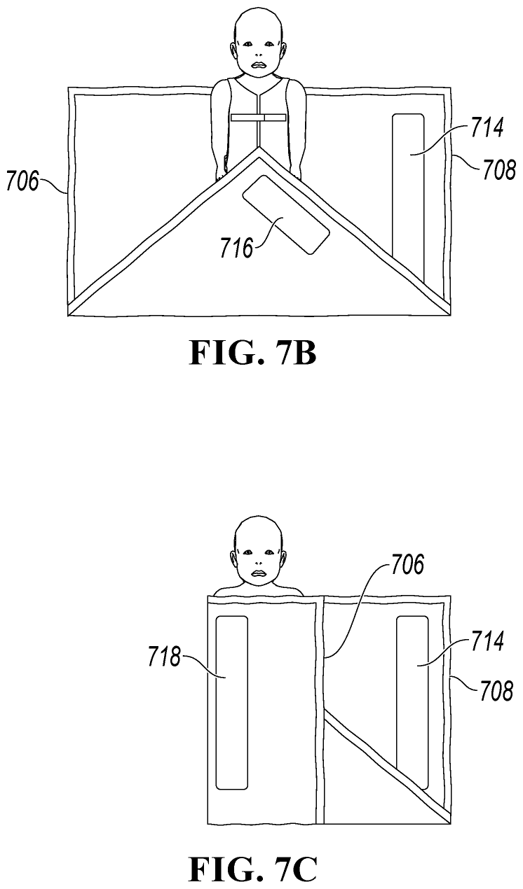

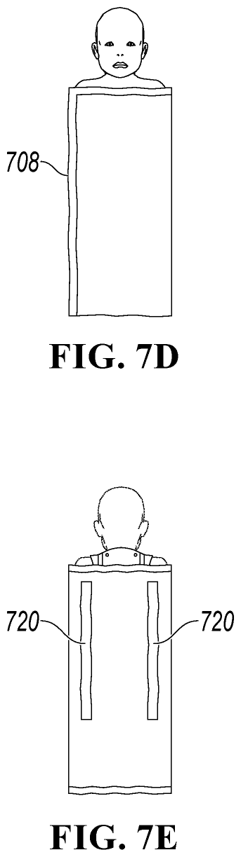

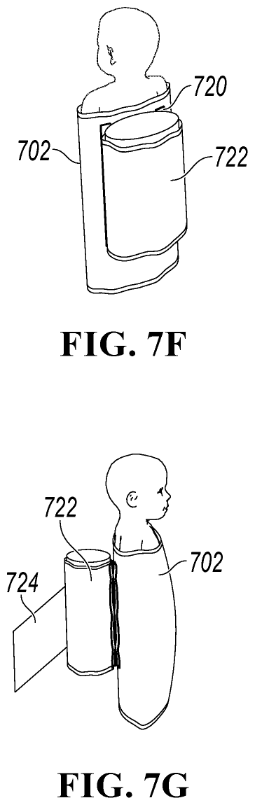

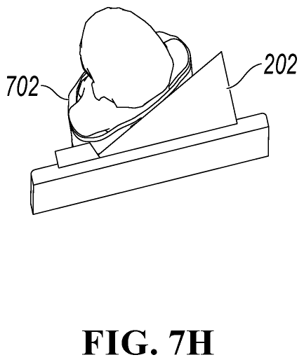

FIG. 7A is a front view of a swaddle wrap 702 in an open position and placed around an infant. FIG. 7B is a front, partially-folded view of the swaddle wrap 702. FIG. 7C is a front, partially-folded view of the swaddle wrap 702. FIG. 7D is a front, folded, view of the swaddle wrap 702. FIG. 7E is a rear, folded, view of the swaddle wrap 702 showing fasteners. Referring to FIGS. 7A-7E collectively, the swaddle wrap 702 includes an upper edge 704, side edges 706, 708, and a lower region 710. As shown by way of example in FIG. 7A, the lower region 710 extends from the side edge 706 and the side edge 708 and comes to an apex below a region where, for example, the feet of an infant would be placed. A first attachment point 712 is positioned inside the side edge 706 and a second attachment point 714 is positioned inside the side edge 708. A third attachment point 716 is positioned in the lower region 710. By way of example, the first attachment point 712, the second attachment point 714, and the third attachment point 716 are illustrated in FIGS. 7A-7C as being hook and pile fasteners; however, in other embodiments, any type of fastener including, for example, snaps, buttons, and interlocking fasteners could be utilized. The swaddle wrap 702 is shown by way of example in FIG. 7A-7G as being used in conjunction with the infant bodice restraint article 602; however, in other embodiments, the swaddle wrap 702 may be utilized without the infant bodice restraint article 602 or with the infant garment 670 that includes the fasteners 614.

Still referring to FIGS. 7A-7E, in use, an infant is positioned on the swaddle wrap 702 infant's head is positioned above the upper edge 704 and the lower region 710 extends below the infant's feet. When the swaddle wrap 702 is used in conjunction with the infant bodice restraint article 602, the fasteners 614 on the infant bodice restraint article 602 are secured to the swaddle wrap 702 thereby fixing a position of the infant relative to the swaddle wrap 702. The lower region 710 is wrapped upwardly over the infant's feet and legs such that the apex of the lower region 710 rests approximately mid-torso on the infant. In various embodiments, the lower region 710 may be permanently folded upwardly to create a pocket into which the infant is placed. The side edge 706 is wrapped snugly across the infant's torso and over the lower region 710. The first attachment point 712 is secured to the third attachment point 716. Wrapping the side edge 706 across the infant reveals a fourth attachment point 718 positioned on an underside of the swaddle wrap 702. The side edge 708 is wrapped snugly across the infant's torso and over the side edge 706 and the lower region 710. The third attachment point 716 is secured to the fourth attachment point 718.

Still referring to FIGS. 7A-7E, fasteners 720 are positioned on a back of the swaddle wrap 702. The fasteners 720 are illustrated by way of example in FIG. 7E as being interlocking fasteners; however in other embodiments, the fasteners 720 could be, for example, snaps, buttons, hook and pile fasteners, or any other type of fastener.

FIG. 7F is a perspective view of the swaddle wrap 702 coupled to a back support 722. The back support 722 is generally cylindrical in shape and is constructed of a soft, pliable material such as for example, polyethylene foam or other appropriate material. In various embodiments, the back support 722 may be wedge-shaped, angled, polygon-shaped, curved, or any combination thereof. Additionally, in various embodiments, the back support may not be solid but may contain a liquid, a gel, or a gas thereby giving a user the ability to adjust a stiffness of the back support 722. The fasteners 720 positioned on the swaddle wrap 702 could be utilized to secure the swaddle wrap to the back support 722; however, in other embodiments, the fasteners 720 could be utilized to secure the swaddle wrap to the infant bodice restraint article 602, a swaddling garment 802, or the infant garment 670 that includes the fasteners 614. During use, the back support 722 maintains the infant in a side-sleeping position prevents the infant from rolling into a face-down position. In various embodiments, the back support 722 may be weighted so as to maintain the infant in a side-sleeping position.

FIG. 7G is a perspective view of the swaddle wrap 702 with the back support 722 coupled to attachable stabilizer wings 724. In various embodiments, the back support 722 may include additional fasteners that facilitate securement of the back support 722 to, for example, the attachable stabilizer wings 724. In various embodiments, however, the back support 722 could be omitted and the swaddle wrap 702 could be secured to the attachable stabilizer wings 724 directly. During use, the attachable stabilizer wings 724 enlarge a support area of the infant and makes the infant resistant to rolling to a face-down position.

FIG. 7H is a perspective view of the swaddle wrap of 702 coupled to the positioning insert 202. The fasteners 720 positioned on the swaddle wrap 702 could be utilized to secure the swaddle wrap to the positioning insert 202. During use, the positioning insert 202 maintains the infant in a side-sleeping position prevents the infant from rolling into a face-down position

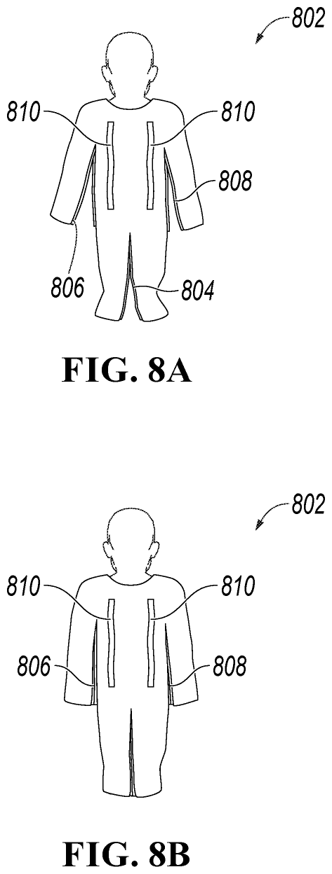

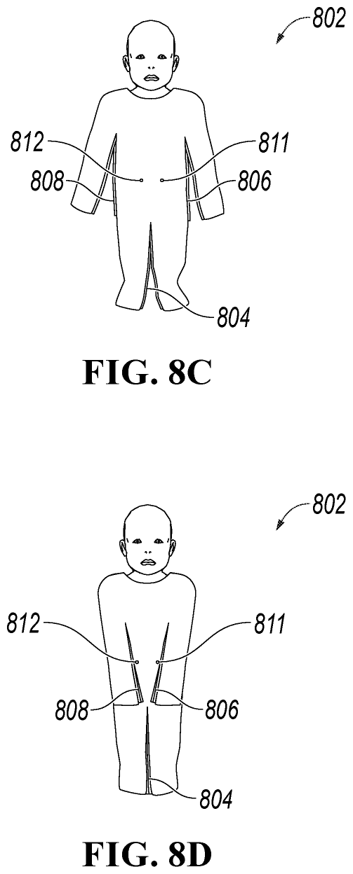

FIG. 8A is a rear view of a swaddling garment 802 with leg fasteners 804 open. FIG. 8B is a rear view of the swaddling garment 802 with the leg fasteners 804 closed. FIG. 8C is a front view of the swaddling garment 802 with the leg fasteners 804 closed. FIG. 8D is a front view of the swaddling garment 802 with the leg fasteners 804 closed. Referring to FIGS. 8A-8D collectively, the swaddling garment 802 includes the leg fasteners 804, left-arm fasteners 806, and right-arm fasteners 808. In various embodiments, the leg fasteners 804, the left-arm fasteners 806, and the right-arm fasteners 808 are interlocking fasteners; however, in other embodiments, the leg fasteners 804, the left-arm fasteners 806, and the right-arm fasteners 808 could be, for example, snaps, buttons, hook and pile fasteners, or any other type of fasteners. The leg fasteners 804, the left-arm fasteners 806, and the right-arm fasteners 808 could also include an elastic or inelastic loop that secures the right sleeve and the left sleeve of the swaddling garment 802. Also, in various embodiments, the leg fasteners 804, the left-arm fasteners 806, and the right-arm fasteners 808 could include a plurality of fasteners. Accessory fasteners 810 are disposed on a back region of the garment 802. In various embodiments, the accessory fasteners 810 are interlocking fasteners; however, in other embodiments, the accessory fasteners 810 could be, for example, snaps, buttons, hook and pile fasteners, or any other type of fasteners.

Still referring to FIGS. 8A-8D, during use, an infant is dressed in the garment 802. If it is desired that the infant be swaddled, the leg fasteners 804 are engaged to secure the infant's legs to each other. Likewise, the left-arm fastener 806 and the right-arm fastener 808 are engaged to secure a left arm and a right arm of the garment 802 to a body of a garment 802, respectively. In other embodiments, the left-arm fastener 806 and the right-arm fastener 808 are utilized to secure the left arm and the right arm of the garment 802 to each other. Additionally, the garment 802 includes a left-sleeve fastener 811 and a right-sleeve fastener 812. During use, the left-sleeve fastener 811 and the right-sleeve fastener 812 may be utilized to fasten the left and right sleeves, respectively, of the garment 802 to a front torso region of the garment 802 in order to further restrict movement of, and thereby swaddle, the infant. In other embodiments, the left-sleeve fastener 811 and the right-sleeve fastener 812 are utilized to secure the left arm and the right arm of the garment 802 to each other.

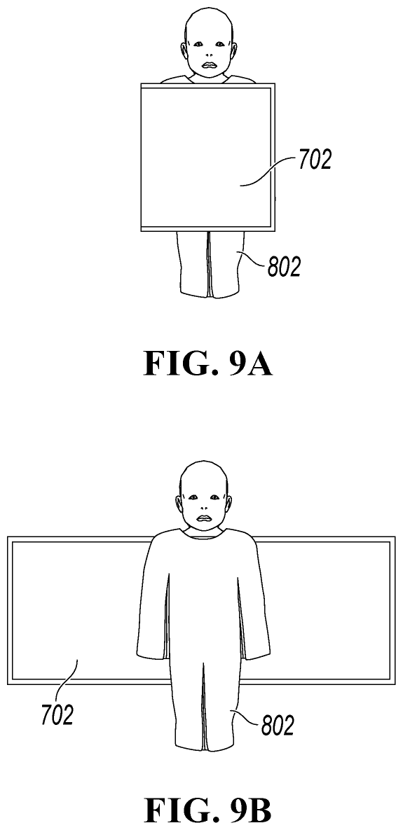

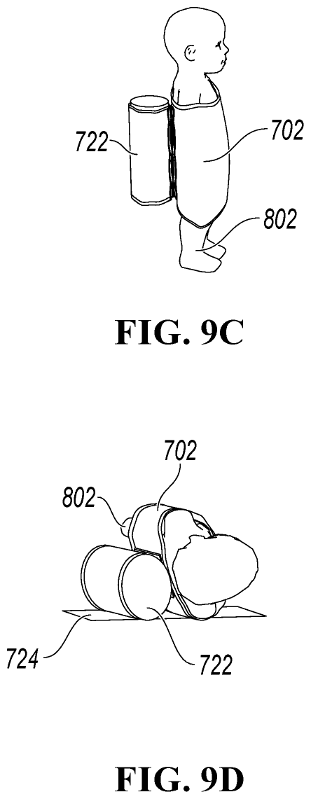

FIGS. 9A-9D illustrate use of the garment 802 with the swaddle wrap 702. In various embodiments, the accessory fasteners 810 of the swaddling garment 802 may be coupled to corresponding fasteners on the swaddle wrap 702. Use of the accessory fasteners 810 fixes a position of the infant relative to the swaddle wrap 702 and reduces movement of the infant within the swaddle wrap 702. The infant, while wearing the garment 802 may then be swaddled with the garment 802 in a manner similar to that described above with respect to FIGS. 8A-8D. FIG. 9C illustrates use of the garment 802 with the swaddle wrap 702 and the back support 722. In various embodiments, the swaddle wrap 702 may be coupled to the back support 722 in a manner similar to that described above with respect to FIG. 7F; however, in other embodiments, the back support 722 could be permanently coupled to the swaddle wrap 702. FIG. 9D illustrates use of the garment 802 with the swaddle wrap 702, the back support 722, and the stabilizer wings 724. In various embodiments, the accessory fasteners 810 may be utilized to couple the garment 802 with accessories such as, for example, the modular base assembly 100, the positioning insert 202, the swaddle wrap 702, the back support 722, the support frame 1002, the stabilizer wings 724, or any combination thereof. In other embodiments, the garment 802 may be permanently coupled to at least one of the modular base assembly 100, the positioning insert 202, the swaddle wrap 702, the back support 722, the stabilizer wings 724 or the support frame 1002.

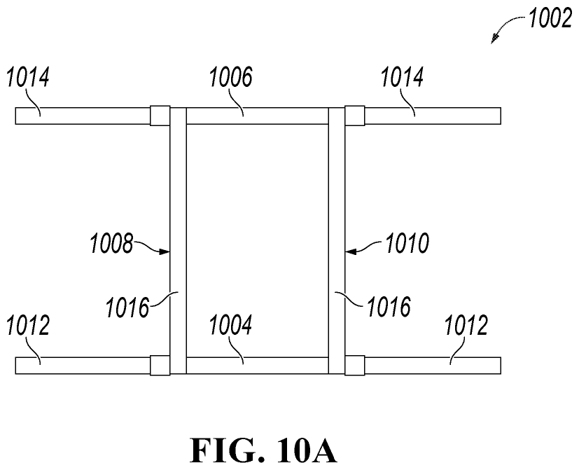

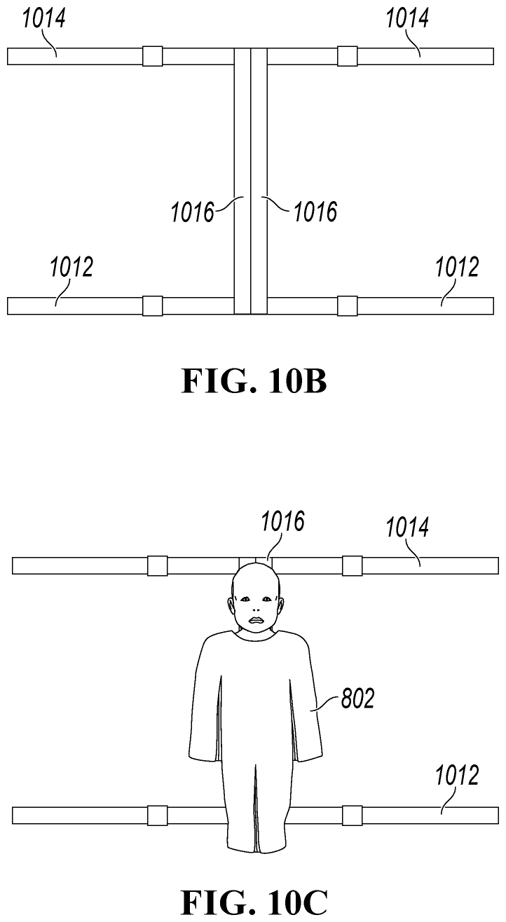

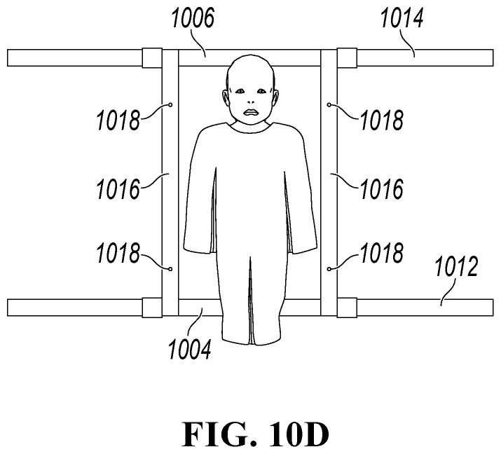

FIG. 10A is a top view of the support frame 1002 in an extended position. FIG. 10B is a top view of the support frame 1002 in a retracted position. FIG. 10C is a top view of the swaddling garment 802 coupled to the back support 722 and the support frame 1002. FIG. 10D is a top view of the swaddling garment 802 coupled to the support frame 1002. Referring to FIGS. 10A-10D collectively, the support frame 1002 includes a first spacing member 1004 and a second spacing member 1006. The support frame 1002 also includes a first leg member 1008 and a second leg member 1010. The first leg member 1008 and the second leg member 1010 are slidably coupled to the first spacing member 1004 and the second spacing member 1006 such that the first leg member 1008 and the second leg member 1010 may be slidably moved towards each other and away from each other. The first leg member 1008 and the second leg member 1010 each include a first leg 1012 arranged generally parallel to a second leg 1014 and an attachment member 1016 that is coupled to the first leg 1012 and the second leg 1014. In various embodiments, the first leg 1012, the second leg 1014, and the attachment member 1016 are integrally formed; however, in other embodiments, the first leg 1012, the second leg 1014, and the attachment member 1016 are separately formed and coupled together.

Still referring to FIGS. 10A-10D, a fastener 1018 is disposed on the attachment member 1016. In various embodiments, the fastener 1018 is an interlocking fastener; however in other embodiments, the fastener 1018 may be, for example, a snap, a button, a hook and pile fastener, or any other appropriate type of fastener. In use, the fastener 1018 may be coupled to the back fastener 810 of the garment 802 or to the back support 722. During use, the first leg member 1008 and the second leg member 1010 may be adjustably moved towards and away from each other thereby adjusting an area of support of the support frame 1002.

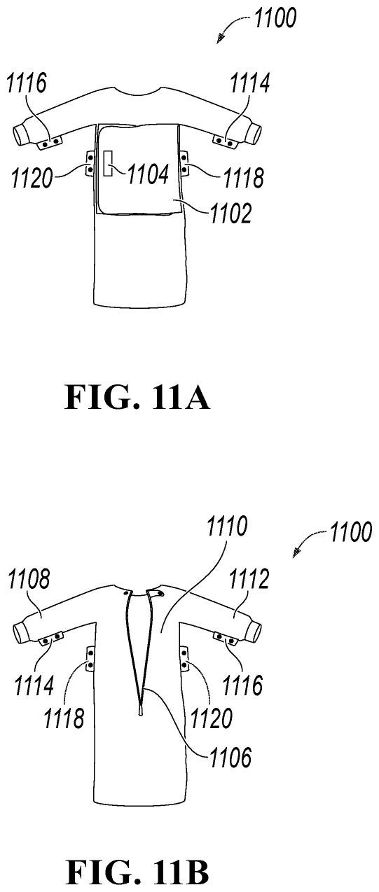

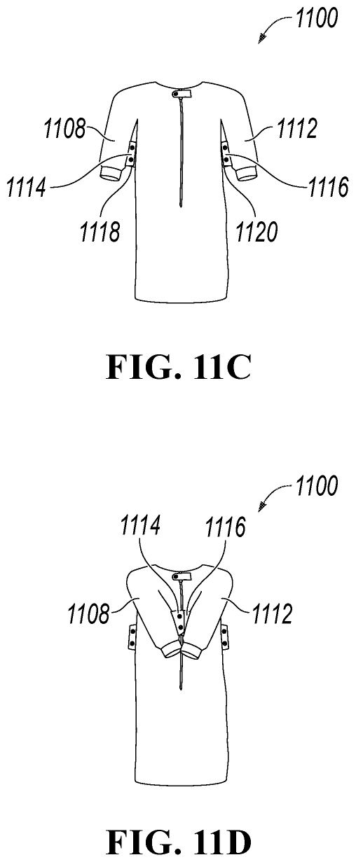

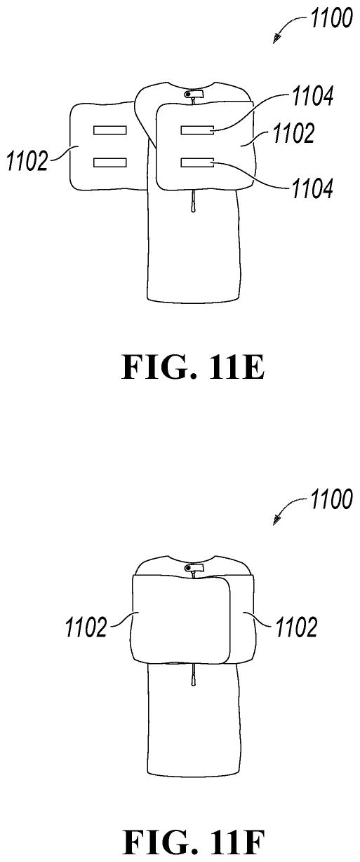

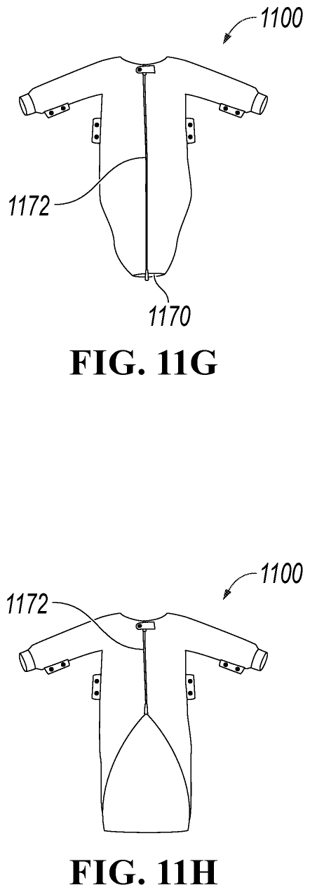

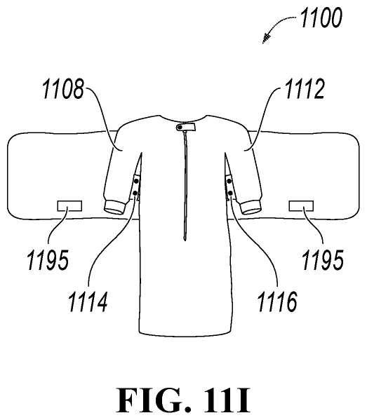

FIG. 11A is a rear view of an infant garment 1100 showing swaddling flaps 1102 in a stored position. In various embodiments, the infant garment 1100 may be, for example, a sleep sack, pouch, gown, or other similar garment. In various embodiments, the infant garment 1100 may be closed along a bottom aspect or, in other embodiments, may be open along the bottom aspect. The swaddling flaps 1102 extend laterally from opposites sides of the infant garment 1100. The swaddling flaps 1102 include a faster 1104 that facilitates joining of the swaddling flaps 1102 to each other. The fastener 1104 is shown by way of example in FIG. 11A as being a hook and pile fastener; however, in other embodiments, the fastener could be, for example, a snap, a button, a zipper, or any other type of fastener. When not in use, the swaddling flaps 1102 wrap across a rear aspect of the infant garment 1100 and are secured to each other via the fastener 1104. Such an arrangement facilitates storage of the swaddling flaps 1102 when not in use and prevents, for example, snagging of the swaddling flaps 1102. By way of example, the infant garment 1100 is illustrated in FIG. 11A as including two swaddling flaps 1102; however, in other embodiments, the infant garment 1100 may include one or more of the swaddling flaps 1102. In various embodiments, the swaddling flaps 1102 could be removable or omitted. As illustrated in FIGS. 11G-11H, in various embodiments, the garment 1100 may include an opening 1170 along a bottom or may include fasteners 1172 to allow the garment 1100 to be opened along the bottom. Such fasteners 1172 in combination with the opening 1170 may, in various embodiments, include, for example, buttons, snaps, zippers, hook-and-loop fasteners, or any other appropriate type of fastener. Use of the fasteners, allows the garment 1100 to fully open in an effort to facilitate, for example, changing of an infant's diaper.

FIG. 11B is a front view of the infant garment 1100 in an open position. The infant garment 1100 includes a vertically-oriented fastener 1106 that facilitates dressing of an infant with the infant garment 1100. In various embodiments, the vertically-oriented fastener 1106 may be, for example, a zipper, a series of buttons, a series of snaps, or other similar fasteners. In various embodiments, the vertically-oriented fastener 1106 may be, for example, a two-way zipper that facilitates opening of the infant garment 1100 from either a top direction or a bottom direction. A first sleeve 1108 extends laterally from a torso portion 1110 of the infant garment 1100 and a second sleeve 1112 extends from the torso portion 1110 in a direction generally opposite to that of the first sleeve 1108. In various embodiments, the infant garment 1100 may include accessory fasteners on a back aspect thereof. The accessory fasteners can be utilized to couple the infant garment 1100 with accessories such as, for example, the modular base assembly 100, the positioning insert 202, the swaddle wrap 702, the back support 722, the support frame 1002, the stabilizer wings 724, or any combination thereof. In various embodiments, the first sleeve 1108 and the second sleeve 1112 may be omitted from the infant garment 1100. In such embodiments, the infant garment 1100 would include first and second openings that, in use, receive the right and left arms, respectively, of the infant and prevent turning of the infant within the infant garment 1100.

Still referring to FIG. 11B, first sleeve fasteners 1114 are disposed on the first sleeve 1108 and second sleeve fasteners 1116 are disposed on the second sleeve 1112. The first sleeve fasteners 1114 and the second sleeve fasteners 1116 are illustrated by way of example in FIGS. 11A-11F as being snaps; however, in other embodiments, the first sleeve fasteners 1114 and the second sleeve fasteners 1116 could be, for example, buttons, zippers, hook and pile fasteners, or any other type of fastener. First side fasteners 1118 are disposed on the torso portion 1110 near the side of the first sleeve 1108 and second side fasteners 1120 are disposed on the torso portion 1110 near the side of second sleeve 1112. The first side fasteners 1118 and the second side fasteners 1120 are illustrated by way of example in FIGS. 11A-11F as being snaps; however, in other embodiments, the first side fasteners 1118 and the second side fasteners 1120 could be, for example, buttons, zippers, hook and pile fasteners, or any other type of fastener. The first sleeve fasteners 1114, the second sleeve fasteners 1116, the first side fasteners 1118, and the second side fasteners 1120 are shown in FIGS. 11A-11E as each including a plurality of fasteners. However, in other embodiments, the first sleeve fasteners 1114, the second sleeve fasteners 1116, the first side fasteners 1118, and the second side fasteners 1120 may include a single fastener each.

FIG. 11C is a front view of the infant garment 1100 showing fastening of the first sleeve 1108 and the second sleeve 1112 to the torso portion 1110. During use, the first sleeve fasteners 1114 may be secured to the first side fasteners 1118 and the second sleeve fasteners 1116 may be secured to the second side fasteners 1120. Such an arrangement restricts movement of the first sleeve 1108 and the second sleeve 1112 and facilitates swaddling of an infant wearing the infant garment 1100.

FIG. 11D is a front view of the infant garment 1100 showing the first sleeve 1108 and the second sleeve 1112 fastened to each other. During use, the first sleeve fasteners 1114 may alternatively be secured to the second sleeve fasteners 1116 thereby securing the first sleeve 1108 to the second sleeve 1112. Such an arrangement reduces a range of movement of the first sleeve 1108 and the second sleeve 1112 and facilitates swaddling of an infant wearing the infant garment 1100. As shown in FIG. 11I, in various other embodiments, the first sleeve fasteners 1114 and the second sleeve fasteners 1116 may alternatively couple to fasteners 1195 disposed on an inner surface of the swaddling flap 1102. Such fasteners 1195 on the inner surface of the swaddling flap 1102 could be either a single fastener or a plurality of fasteners including, for example, snaps, buttons, hook and loop fasteners, or any other type of fastener.

FIG. 11E is a front view of the infant garment 1100 showing the swaddling flaps 1102 partially secured. FIG. 11F is a front view of the infant garment 1100 showing the swaddling flaps 1102 secured to each other in front of the infant garment 1100. Referring to FIGS. 11E and 11F collectively, during use the swaddling flaps 1102 may be wrapped around a front aspect of the torso portion 1110 and secured to each other via the fastener 1104. Use of the swaddling flaps 1102 further restricts movement of the first sleeve 1108 and the second sleeve 1112 and facilitates swaddling of an infant wearing the infant garment 1100.

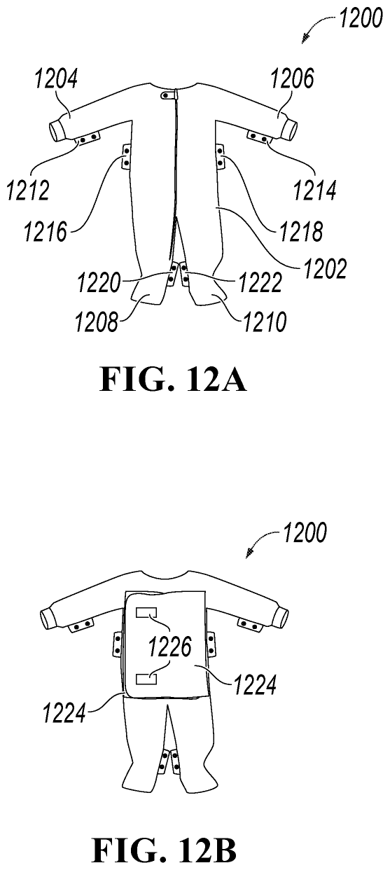

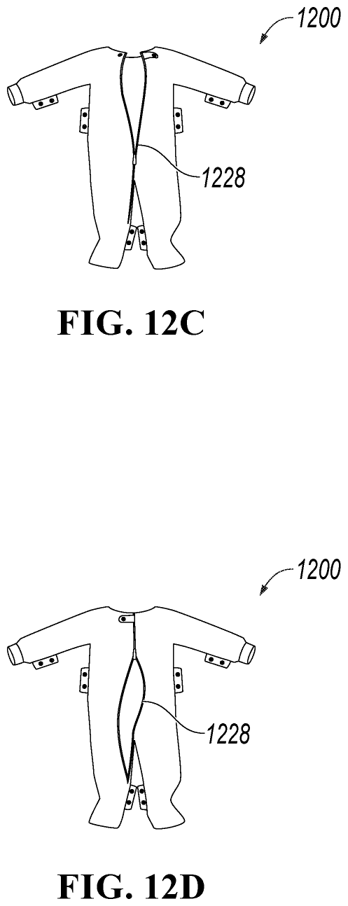

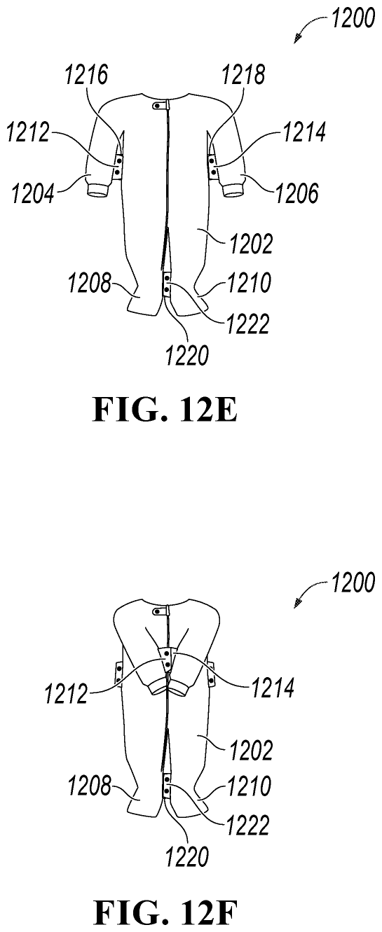

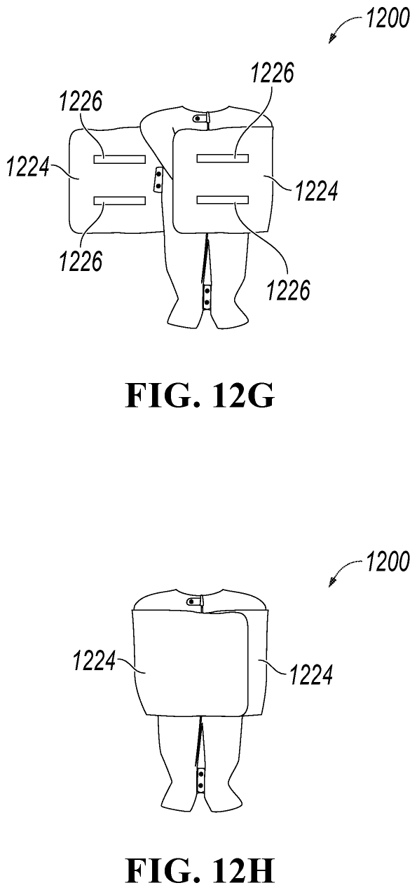

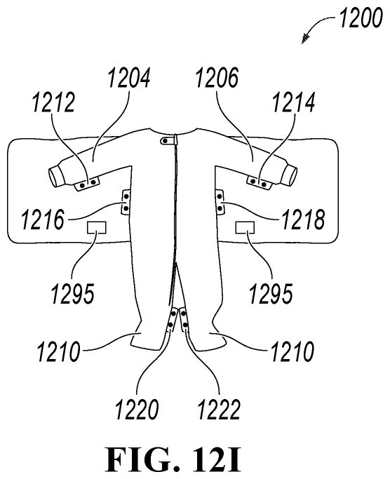

FIG. 12A is a front view of an infant garment 1200. The infant garment 1200 includes a torso portion 1202. A first sleeve 1204 extends from the torso portion 1202 and a second sleeve 1206 extends from the torso portion 1202 on a side opposite the first sleeve 1204. A first leg region 1208 and a second leg region 1210 extend from a bottom aspect of the torso portion 1202. First sleeve fasteners 1212 are disposed on the first sleeve 1204 and second sleeve fasteners 1214 are disposed on the second sleeve 1206. The first sleeve fasteners 1212 and the second sleeve fasteners 1214 are illustrated by way of example in FIGS. 12A-12H as being snaps; however, in other embodiments, the first sleeve fasteners 1212 and the second sleeve fasteners 1214 could be, for example, buttons, zippers, hook and pile fasteners, or any other type of fastener. First side fasteners 1216 are disposed on the torso portion 1202 on a side near the first sleeve 1204 and second side fasteners 1218 are disposed on the torso portion 1202 on a side near the second sleeve 1206. The first side fasteners 1216 and the second side fasteners 1218 are illustrated by way of example in FIGS. 12A-12H as being snaps; however, in other embodiments, the first side fasteners 1216 and the second side fasteners 1218 could be, for example, buttons, zippers, hook and pile fasteners, or any other type of fastener. First leg fasteners 1220 are disposed on the first leg region 1208 and second leg fasteners 1222 are disposed on the second leg region 1210. The first leg fasteners 1220 and the second leg fasteners 1222 are illustrated by way of example in FIGS. 12A-12H as being snaps; however, in other embodiments, the first leg fasteners 1220 and the second leg fasteners 1222 could be, for example, buttons, zippers, hook and pile fasteners, or any other type of fastener. The first sleeve fasteners 1212, the second sleeve fasteners 1214, the first side fasteners 1216, and the second side fasteners 1218 are shown in FIGS. 12A-12G as each including a plurality of fasteners. However, in other embodiments, the first sleeve fasteners 1212, the second sleeve fasteners 1214, the first side fasteners 1216, the second side fasteners 1218, the first leg fasteners 1220, and the second leg fasteners 1222 may include a single fastener each. In various embodiments, the first sleeve 1204 and the second sleeve 1206 may be omitted from the infant garment 1200. In such embodiments, the infant garment 1200 would include first and second openings that, in use, receive the right and left arms, respectively, of the infant and prevent turning of the infant within the infant garment 1200. In various embodiments, the first leg region 1208 and the second leg region 1210 could be omitted. In such embodiments, infant garment 1300 could include openings for the infant's legs.

FIG. 12B is a rear view of the infant garment of 1200 showing swaddling flaps 1224 in a stored position. The swaddling flaps 1224 extend laterally from opposite sides of the torso portion 1202. The swaddling flaps 1224 include a fastener 1226 that facilitates joining the swaddling flaps 1224 to each other. The fastener 1226 is shown by way of example in FIGS. 12A-12H as being a hook and pile fastener; however, in other embodiments, the fastener 1226 could be, for example, a snap, a button, a zipper, or any other type of fastener. When not in use, the swaddling flaps 1224 wrap across a rear aspect of the infant garment 1200 and are secured to each other via the fastener 1226. Such an arrangement facilitates storage of the swaddling flaps 1224 when not in use and prevents, for example, snagging of the swaddling flaps 1224. In various embodiments, the swaddling flaps 1224 could be removable or omitted. By way of example, the infant garment 1200 is illustrated in FIG. 12A as including two swaddling flaps 1224; however, in other embodiments, the infant garment 1200 may include one or more of the swaddling flaps 1224. In various embodiments, the infant garment 1200 may include accessory fasteners on a back aspect thereof. The accessory fasteners can be utilized to couple the infant garment 1200 with accessories such as, for example, the modular base assembly 100, the positioning insert 202, the swaddle wrap 702, the back support 722, the support frame 1002, the stabilizer wings 724, or any combination thereof.

FIG. 12C is a front view of the infant garment 1200 showing a vertically-oriented fastener 1228 in an open position. FIG. 12D is a front view of the infant garment 1200 showing the vertically-oriented fastener 1228 in an alternative open position. Referring to FIGS. 12C and 12D collectively, the infant garment 1200 includes the vertically-oriented fastener 1228 that facilitates dressing of an infant with the infant garment 1200. In various embodiments, the vertically-oriented fastener 1228 may be, for example a zipper, a series of buttons, a series of snaps, or other similar fasteners. By way of example, the vertically-oriented fastener 1228 is shown in FIGS. 12C and 12D as a two-way zipper that facilitates opening of the infant garment 1100 from either a top direction (FIG. 12C) or a bottom direction (FIG. 12D).

FIG. 12E is a front view of the infant garment 1200 showing the first sleeve 1204 and the second sleeve 1206 joined to the torso portion 1202. During use, the first sleeve fasteners 1212 may be secured to the first side fasteners 1216 and the second sleeve fasteners 1214 may be secured to the second side fasteners 1218 in an effort to restrict a range of movement of the first sleeve 1204 and the second sleeve 1206. Additionally, the first leg fasteners 1220 may be secured to the second leg fasteners 1222 in order to restrict a range of movement of the first leg region 1208 and the second leg region 1210. Such an arrangement restricts movement of the first sleeve 1204, the second sleeve 1206, the first leg region 1208, and the second leg region 1210 and facilitates swaddling of an infant wearing the infant garment 1200. Based on preference of the parents and the infant, the first leg fasteners 1220 and the second leg fasteners 1222 could be left unattached such that the infant's legs are unrestricted while restricting arm movement. In various embodiments, the first leg fasteners 1220 and the second leg fasteners 1222 could be omitted.

FIG. 12F is a front view of the infant garment 1200 showing the first sleeve 1204 and the second sleeve 1206 joined to each other. During use, the first sleeve fasteners 1212 may alternatively be secured to the second sleeve fasteners 1214 thereby securing the first sleeve 1204 to the second sleeve 1206. Such an arrangement reduces a range of movement of the first sleeve 1204 and the second sleeve 1206 and facilitates swaddling of an infant wearing the infant garment 1200. Based on preference of the parents and the infant, the first leg fasteners 1220 and the second leg fasteners 1222 could be left unattached such that the infant's legs are unrestricted while restricting arm movement. As shown in FIG. 12I, in various other embodiments, the first sleeve fasteners 1212 and the second sleeve fasteners 1214 may alternatively couple to fasteners 1295 disposed on an inner surface of the swaddling flap 1224. Such fasteners 1295 on the inner surface of the swaddling flap 1224 could be either a single fastener or a plurality of fasteners including, for example, snaps, buttons, hook and loop fasteners, or any other type of fastener.

FIG. 12G is a front view of the infant garment 1200 showing the swaddling flaps 1224 partially secured. FIG. 12H is a front view of the infant garment 1200 showing the swaddling flaps 1224 secured to each other in front of the infant garment 1200. Referring to FIGS. 12G-12H collectively, during use the swaddling flaps 1224 may be wrapped around a front aspect of the torso portion 1202 and secured to each other via the fastener 1226. Use of the swaddling flaps 1224 further restricts movement of the first sleeve 1204 and the second sleeve 1206 and facilitates swaddling of an infant wearing the infant garment 1200. In various embodiments, the infant garment 1200 may include accessory fasteners on a back aspect thereof. The accessory fasteners can be utilized to couple the infant garment 1200 with accessories such as, for example, the modular base assembly 100, the positioning insert 202, the swaddle wrap 702, the back support 722, the support frame 1002, the stabilizer wings 724, or any combination thereof.

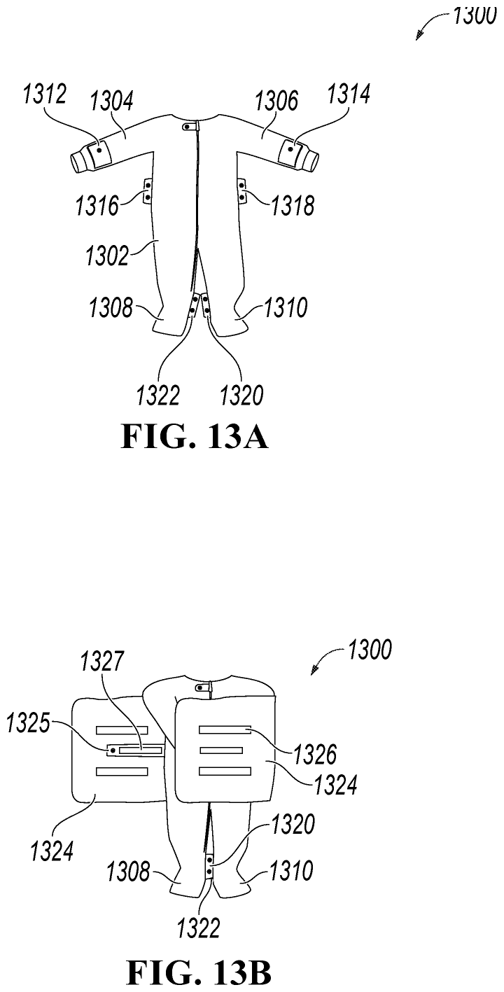

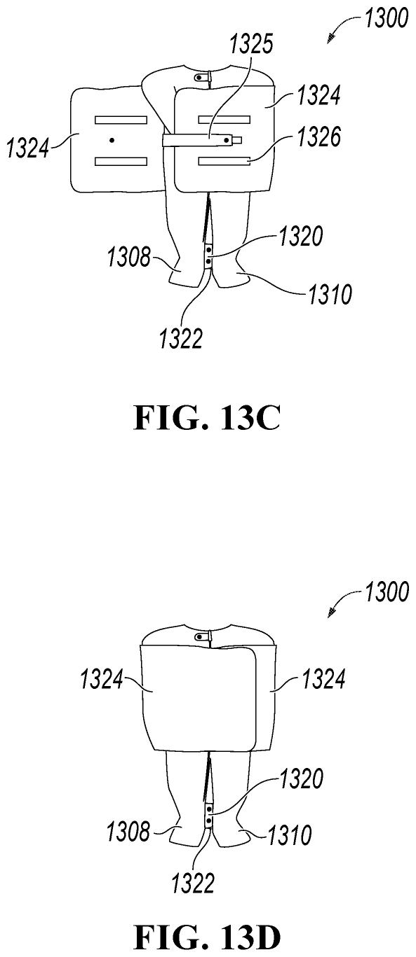

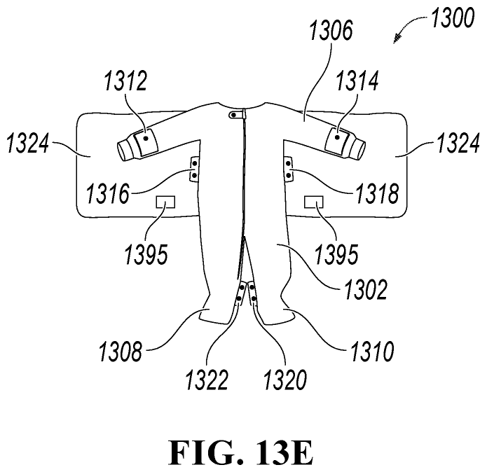

FIG. 13A is a front view of an infant garment 1300. The infant garment 1300 includes a torso portion 1302. A first sleeve 1304 extends from the torso portion 1302 and a second sleeve 1306 extends from the torso portion 1302 on a side opposite the first sleeve 1304. A first leg region 1308 and a second leg region 1310 extend from a bottom aspect of the torso portion 1302. First sleeve fastener 1312 is disposed on the first sleeve 1304 and second sleeve fastener 1314 is disposed on the second sleeve 1306. The first sleeve fastener 1312 and the second sleeve fastener 1314 are illustrated by way of example in FIGS. 13A-13D as being snaps; however, in other embodiments, the first sleeve fastener 1312 and the second sleeve fastener 1314 could be, for example, buttons, zippers, hook and pile fasteners, or any other type of fastener. By way of example, the first sleeve fastener 1312 and the second sleeve fastener 1314 are illustrated as a single snap; however, in other embodiments, the first sleeve fastener 1312 and the second sleeve fastener 1314 could be a plurality of fasteners or a combination of any of the above-mentioned types of fasteners.

Still referring to FIG. 13A, a first side fastener 1316 is disposed on the torso portion 1302 on a side near the first sleeve 1304 and second side fastener 1318 is disposed on the torso portion 1302 on a side near the second sleeve 1306. The first side fastener 1316 and the second side fastener 1318 are illustrated by way of example in FIGS. 13A-13D as being snaps; however, in other embodiments, the first side fastener 1316 and the second side fastener 1318 could be, for example, buttons, zippers, hook and pile fasteners, or any other type of fastener. During use, the first sleeve fastener 1312 couples to the first side fastener 1316 and the second sleeve fastener 1314 couples to the second side fastener 1318 in an effort to secure the first sleeve 1304 and the second sleeve 1306 and reduce a range of motion of the infant. As shown in FIG. 13E, in various other embodiments, the first sleeve fastener 1312 and the second sleeve fastener 1314 may alternatively couple to fasteners 1395 disposed on an inner surface of the swaddling flap 1324. Such fasteners 1395 on the inner surface of the swaddling flap 1324 could be either a single fastener or a plurality of fasteners including, for example, snaps, buttons, hook and loop fasteners, or any other type of fastener. In other embodiments, the first sleeve fastener 1312 and the second sleeve fastener 1314 may couple to each other and not to the first side fastener 1316 and the second side fastener 1318, respectively. Coupling of the first sleeve fastener 1312 to the second sleeve fastener 1314 reduces a range of motion of the first sleeve 1304 and the second sleeve 1306 and secures the infant.

Still referring to FIG. 13A, first leg fastener 1320 is disposed on the first leg region 1308 and second leg fastener 1322 is disposed on the second leg region 1310. The first leg fastener 1320 and the second leg fastener 1322 are illustrated by way of example in FIGS. 13A-13D as being snaps; however, in other embodiments, the first leg fastener 1320 and the second leg fastener 1322 could be, for example, buttons, zippers, hook and pile fasteners, or any other type of fastener. In various embodiments, the first leg fastener 1320 and the second leg fastener 1322 could include a plurality of fasteners. In various embodiments, the first leg fastener 1320 and the second leg fastener 1322 could be coupled together to restrict leg movement; however, in other embodiments, the first leg fastener 1320 and the second leg fastener 1322 could be left unattached so as to leave unrestricted leg movement. In various embodiments, the first leg fastener 1320 and the second leg fastener 1322 could be omitted. In various embodiments, the first sleeve 1304 and the second sleeve 1306 may be omitted from the infant garment 1300. In such embodiments, the infant garment 1300 would include first and second openings that, in use, receive the right and left arms, respectively, of the infant and prevent turning of the infant within the infant garment 1300. In various embodiments, the first leg region 1308 and the second leg region 1310 could be omitted. In such embodiments, infant garment 1300 could include openings for the infant's legs.

FIG. 13B is a front view of the infant garment 1300 illustrating swaddling flaps 1324 and tether 1325. FIG. 13C is a front view of the infant garment 1300 illustrating the tether 1325 securing the swaddling flaps 1324. Referring to FIGS. 13B-13C collectively, the swaddling flaps 1324 extend laterally from opposite sides of the torso portion 1302. The swaddling flaps 1324 include a fastener 1326 that facilitates joining the swaddling flaps 1324 to each other. The fastener 1326 is shown by way of example in FIGS. 13A-13D as being a hook and pile fastener; however, in other embodiments, the fastener 1326 could be, for example, a snap, a button, a zipper, or any other type of fastener. When not in use, the swaddling flaps 1324 wrap across a rear aspect of the infant garment 1300 and are secured to each other via the fastener 1326. Such an arrangement facilitates storage of the swaddling flaps 1324 when not in use and prevents, for example, snagging of the swaddling flaps 1324. A tether 1325 extends from the torso portion 1302 and includes a fastener 1327. During use, the tether 1325 is coupled, via the fastener 1327, to the swaddling flaps 1324 and further secures the swaddling flaps 1324 during swaddling.

FIG. 13D is a front view of the infant garment 1300 illustrating the swaddling flaps 1324 being secured. During use the swaddling flaps 1324 may be wrapped around a front aspect of the torso portion 1302 and secured to each other via the fastener 1326. Use of the swaddling flaps 1324 further restricts movement of the first sleeve 1304 and the second sleeve 1306 and facilitates swaddling of an infant wearing the infant garment 1300. By way of example, the infant garment 1300 is illustrated in FIG. 13D as including two swaddling flaps 1324; however, in other embodiments, the infant garment 1300 may include one or more of the swaddling flaps 1324. In various embodiments, the infant garment 1300 may include accessory fasteners on a back aspect thereof. The accessory fasteners can be utilized to couple the infant garment 1300 with accessories such as, for example, the modular base assembly 100, the positioning insert 202, the swaddle wrap 702, the back support 722, the stabilizer wings 724, the support frame 1002, or any combination thereof.

Depending on the embodiment, certain acts, events, or functions of any of the algorithms, methods, or processes described herein can be performed in a different sequence, can be added, merged, or left out altogether (e.g., not all described acts or events are necessary for the practice of the algorithms, methods, or processes). Moreover, in certain embodiments, acts or events can be performed concurrently, e.g., through multi-threaded processing, interrupt processing, or multiple processors or processor cores or on other parallel architectures, rather than sequentially. Although certain computer-implemented tasks are described as being performed by a particular entity, other embodiments are possible in which these tasks are performed by a different entity.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices or algorithms illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

1. An infant garment, comprising:
a body adapted to surround a torso of an infant;
a first sleeve coupled to the body;
a second sleeve coupled to the body; and
a swaddle wrap coupled to the body and that, when in use, compresses the first sleeve and the second sleeve to the body and restricts movement.

2. The infant garment of claim 1, comprising:
a first body fastener;
a second body fastener;
a first sleeve fastener disposed on the first sleeve;
a second sleeve fastener disposed on the second sleeve;
wherein the first body fastener couples to the first sleeve fastener in order to restrict movement of the first sleeve; and
wherein the second body fastener couples to the second sleeve fastener in order to restrict movement of the second sleeve.

3. The infant garment of claim 1, comprising:
a first sleeve fastener disposed on the first sleeve;
a second sleeve fastener disposed on the second sleeve;
a plurality of fasteners disposed on the swaddle wrap; and
wherein the first sleeve fastener and the second sleeve fastener couple to the plurality of fasteners in order to restrict movement of the first sleeve and the second sleeve.

4. The infant garment of claim 3, wherein the first sleeve fastener and the second sleeve fastener couple to each other in order to restrict movement of the first sleeve and the second sleeve.

5. The infant garment of claim 1, wherein the swaddle wrap is moveable to a stored position.

6. The infant garment of claim 1, wherein the swaddle wrap is selectively removeable from the body.

7. The infant garment of claim 1, comprising accessory fasteners positioned on the body, wherein the accessory fasteners couple to at least one of a base assembly, a back support, and a stabilizer wing.

8. (canceled)

9. The infant garment of claim 7, wherein the back support, when coupled to the infant garment, places an infant's back on an angular plane that encourages the infant's head to a side position.

10. The infant garment of claim 7, wherein the base assembly is selectively coupled to at least one of a swing, a cradle, a rocker, a bassinette, and a play yard.

11. The infant garment of claim 7, wherein:
the base assembly comprises a sleeping surface and a stabilizer wing; and
the accessory fasteners couple to at least one of the base assembly and the sleeping surface.

12. (canceled)

13. The infant garment of claim 7, wherein the base assembly comprises a positioning insert.

14. The infant garment of claim 1, comprising:
a first leg fastener disposed on a first leg of the infant garment;
a second leg fastener disposed on a second leg of the infant garment; and
wherein the first leg fastener couples the second leg fastener to restrict movement of the first leg and the second leg.

15. The infant garment of claim 1, comprising a pouch region for receiving legs of an infant.

16. An infant garment, comprising:
a body for receiving arms, legs, and torso of an infant; and
a fastener disposed on a body of the infant garment;
a swaddle wrap, the swaddle wrap comprising:
a fastener that engages with the fastener of the infant garment thereby coupling the swaddle wrap to the infant garment;
an upper edge;
a first side edge;
a second side edge;
a lower region;
a first attachment point disposed on the first side edge;
a second attachment point disposed on the second side edge;
a third attachment point disposed in the lower region; and
wherein the first attachment point, the second attachment point, and the third attachment point are selectively coupled to each other to swaddle an infant wearing the infant garment.

17. (canceled)

18. The infant garment of claim 16, wherein the fastener couples to at least one of a base assembly, a back support, a swaddle wrap, and a stabilizer wing.

19. The infant garment of claim 18, wherein:
the base assembly comprises a sleeping surface; and
the fastener couples to at least one of the base assembly and the sleeping surface.

20. The infant garment of claim 18, wherein the base assembly comprises a stabilizer wing that is extendable from the base assembly and selectively removable from the base assembly.

21-22. (canceled)

23. The infant garment of claim 18, wherein the base assembly comprises an positioning insert that is selectively removable from the base assembly.

24. The infant garment of claim 23, wherein the base assembly comprises a shoulder rest.

25. (canceled)

26. An infant positioning system, comprising:
a garment having a body adapted to surround a torso of an infant, the garment further comprising:
a first sleeve coupled to the body;
a second sleeve coupled to the body;
a first sleeve fastener disposed on the first sleeve;
a second sleeve fastener disposed on the second sleeve;
a swaddle wrap coupled to the body and that, when in use, compresses the first sleeve and the second sleeve to the body and restricts movement;
wherein the first sleeve fastener and the second sleeve fastener restrict movement of the first sleeve and the second sleeve; and
a modular base assembly having an angular sleeping surface for securement of the garment thereon with the infant therein.

27. (canceled)
